(19) United States
(12) Patent Application Publication
(10) Pub. No.: US 2007/0299433 A1
(43) Pub. Date: Dec. 27, 2007

(54) Barrett's Esophagus Cryogenic Ablation System

(75) Inventors: Richard S. Williams, Redwood City, CA (US); Peter Garcia-Meza, San Francisco, CA (US)

Correspondence Address: HAYNES BEFFEL & WOLFELD LLP, P O BOX 366, HALF MOON BAY, CA, 94019, US

(73) Assignee: C2 Therapeutics, Redwood City, CA (US)

(21) Appl. No.: 11/768,746

(22) Filed: Jun. 26, 2007

Related U.S. Application Data
(60) Provisional application No. 60/805,965, filed on Jun. 27, 2006.

(51) Int. Cl. A61B 18/02; A61M 25/10

(52) U.S. Cl. 606/21; 604/99.01

(57) ABSTRACT

A medical device for treating esophageal tissue comprises a catheter, a balloon, placeable within the esophagus of the patient, and a refrigerant. The refrigerant is deliverable into the interior of the balloon so to place the balloon into an expanded, cooled state so that the balloon can press against and cool esophageal tissue. In other examples the medical device may include means for limiting radial expansion of the balloon.

### CROSS-REFERENCE TO RELATED APPLICATION

This application claims the benefit of U.S. provisional patent application No. 60/805,965 filed 27 Jun. 2006 and having the same title, attorney docket number WILL 1001-1.

### BACKGROUND OF THE INVENTION

Barrett's Esophagus is a pre-cancerous condition of the esophagus typically often associated with gastric reflux disease (GERD). Although GERD can be medically controlled, Barrett's Esophagus does not spontaneous resolve once the GERD has abated. However, it has been shown that if Barrett's Esophagus is ablated, the normal esophagus lining can be restored and therefore lowering the risk of developing esophageal cancer.

A variety of techniques have been evaluated for ablation of this condition. These techniques include photodynamic therapy, endoscopic resection of the lining of the esophagus, and ablation using a variety of energy sources such as argon plasma coagulation (APC), radio-frequency (RF) and cryogenic via a direct spray of liquid nitrogen.

### BRIEF SUMMARY OF THE INVENTION

An example of a medical device for treating atypical esophageal tissue comprises a placement catheter, an expandable balloon and a supply of refrigerant. The placement catheter comprises a distal portion and a lumen. The expandable balloon is mounted to the distal portion of the placement catheter to create a catheter assembly. The balloon has an interior coupled to the lumen. The balloon is placeable within the esophagus of the patient. The supply of a refrigerant is deliverable through the lumen and into the interior of the balloon so to place the balloon into an expanded, cooled state so that the balloon can press against and cool esophageal tissue. In other examples the medical device may include means for limiting expansion of the balloon. Such expansion means may include (a) a radial expansion-limiting tubular braid surrounding the balloon and having a first end fixed to the catheter assembly and a second end movable along the placement catheter, and (b) means for fixing the second end at a chosen position relative to the placement catheter so to limit radial expansion of the tubular braid thereby preventing overexpansion of the balloon.

An example of a method for treating atypical esophageal tissue is carried out as follows. A catheter assembly is selected. The catheter assembly includes a placement catheter comprising a distal portion, a lumen and an expandable balloon mounted to the distal portion, the balloon having an interior coupled to the lumen. The balloon is placed within the esophagus of a patient. The balloon is positioned at a target site having atypical esophageal tissue. A refrigerant is delivered through the lumen and into the interior of the balloon thereby (a) expanding the balloon to a chosen size, and (b) cooling the balloon so that the balloon presses against and cools the atypical esophageal tissue. In other examples a radial expansion limiting step may comprise the following steps. The balloon may be surrounded with a radial expansion-limiting tubular braid and having a first end fixed to the catheter assembly and a second end movable along the placement catheter. The second end may be fixed to the catheter assembly at a chosen position thereby (a) limiting radial expansion of the tubular braid, and (b) preventing overexpansion of the balloon. Other examples may also comprise cooling only a portion of the esophageal tissue in contact with the expanded balloon.

Other features, aspects and advantages of the present invention can be seen on review the figures, the detailed description, and the claims which follow.

### BRIEF DESCRIPTION OF THE DRAWINGS

FIG. 1 is a simplified overall view of one example of a medical device made according to the invention with elements of other examples shown in dashed lines;

FIG. 2 is an enlarged, simplified cross-sectional view of the distal portion of the medical device of FIG. 1;

FIG. 3 illustrates the balloon of FIG. 1 in a collapsed state with a braided structure covering the balloon and a distal portion of the placement catheter;

FIG. 4 illustrates the structure of FIG. 3 with the balloon in a radially expanded state and showing how the braided structure shortens longitudinally as the balloon expands;

FIG. 5 is a simplified cross-sectional view of another example in which two balloons are inflated generally equal amounts within an esophagus;

FIG. 6 is a schematic side illustration the balloon structure of FIG. 5; and

FIG. 7 is a view similar to that of FIG. 5 but with only one balloon inflated.

### DETAILED DESCRIPTION OF THE INVENTION

The following description of the invention will typically be with reference to specific structural embodiments and methods. It is to be understood that there is no intention to limit the invention to the specifically disclosed embodiments and methods but that the invention may be practiced using other features, elements, methods and embodiments. Preferred embodiments are described to illustrate the present invention, not to limit its scope, which is defined by the claims. Those of ordinary skill in the art will recognize a variety of equivalent variations on the description that follows.

All of the techniques listed above suffer from ‘usability’ drawbacks. Photodynamic therapy renders the patient susceptible to sunlight for several months following treatment and has a high procedural complication rate. Mechanical resection is training intensive and may not achieve 100% removal of the condition. Ablation techniques such as APC only treat a small area at a time and controlling the depth of ablation is difficult. Current RF ablation techniques require precise sizing of the treatment catheter and require another console for the physician to operate. The direct spray of liquid nitrogen can be training intensive and is very operator dependent; this system also requires an additional console and a constant supply of liquid nitrogen.

The present invention addresses many of the limitations of the current technologies. Embodiments of the invention typically include a self sizing treatment catheter connected to a refrigerant delivery handle. The invention is particularly useful for treating Barrett's esophagus but may also be useful for treating other esophageal tissues, typically by cryogenic ablation of the atypical tissue.

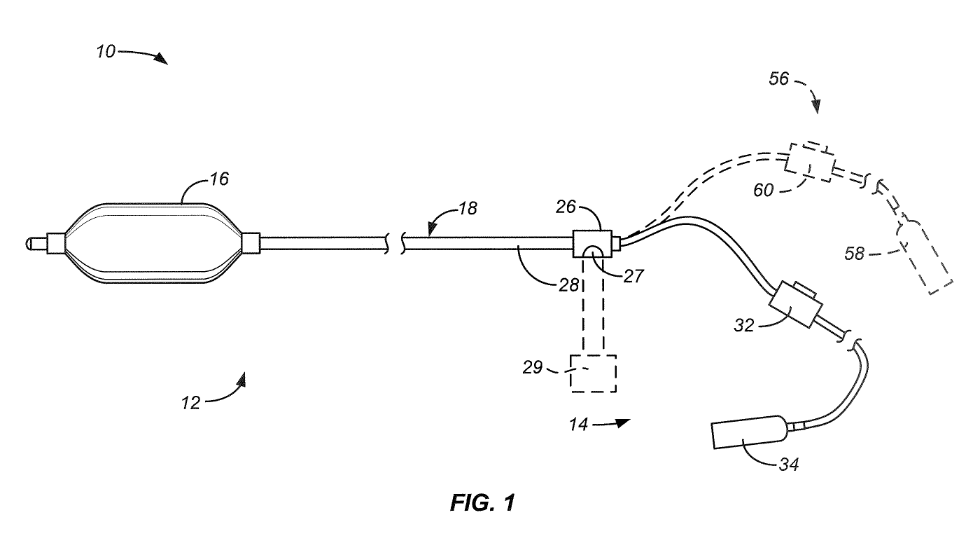

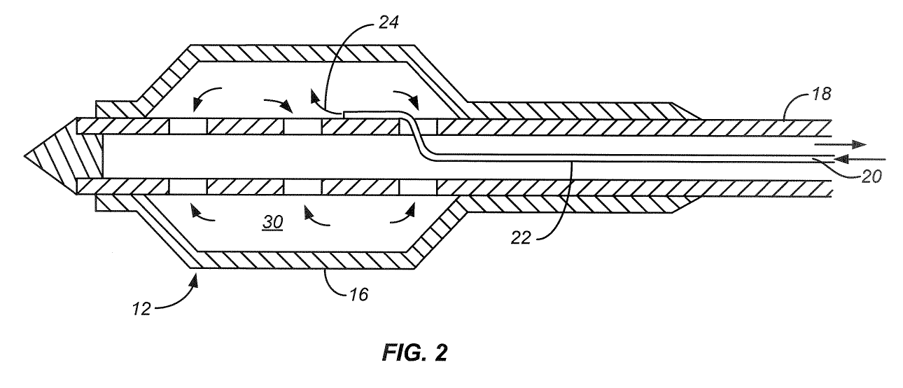

According to some embodiments of the invention, see FIGS. 1 and 2, the medical device 10 comprises a catheter assembly 12 and a refrigerant supply 14. The catheter assembly 12 comprises a balloon 16, preferably an elastomeric material such as polyurethane or silicone, mounted to a placement catheter or shaft 18. In one embodiment the balloon 16 will be capable of producing an inflated diameter of between 15-45 mm. In another embodiment, multiple balloon sizes may be required to cover the desired range of esophagus sizes; in this embodiment, it is desirable to have individual balloon diameters that are variable by at least 2 mm. For example, 6 different sizes could be developed to cover the complete range of 15-45 mm in which case each size covers a 5 mm range. The balloon length may be 10-100 mm. The shaft 18 may comprise a plastic such as polyurethane such that the balloon may be appropriately bonded to the shaft; other appropriate, biocompatible materials such as PEBAX and polyethylene may also be used. The shaft 18 will be less than 8-Fr in order to be compatible with a conventional diagnostic endoscope, which typically has an accessory channel size of 2.8 mm. However, larger shaft sizes up to 11-Fr may be utilized for catheters designed for conventional therapeutic endoscopes. Shaft 18 may include a delivery lumen 20, which may be used for delivery of the refrigerant, running through, and which may be concentric with, the shaft 18 and may have an inner diameter of, for example, 0.004-0.025″ (0.10-0.71 mm). In some embodiments, as disclosed in FIG. 2, the delivery lumen 20 may comprise a separate delivery tube 22 passing through the interior of the shaft 18; all or part of delivery tube 22 could also pass along the exterior of shaft 18. This delivery tube 22 may comprise a high-strength material such as polyimide. In other embodiments, the shaft itself will define at least a portion of the refrigerant delivery lumen.

A fluid saturated liquid/gas refrigerant 24, indicated by arrows 24 in FIG. 2, such as nitrous oxide or a hydrofluorocarbon, is provided from the refrigerant supply 14 through a manifold 26 at the proximal end 28 of the shaft 18, through the delivery lumen 20 and into the interior 30 of the balloon. As shown in FIG. 1, one example of a refrigerant supply 14 of medical device 10 comprises a flow control device 32 which may be hand-held, coupled to a disposable cylinder 34 of refrigerant. The size of the cylinder 34 may be between 10 to 50 cubic centimeters in volume. The refrigerant supply 14 may be integral to the catheter assembly 12 or stand-alone. The refrigerant 24 will typically be continuously injected, at room temperature or warmer, into the delivery lumen 20 and in some embodiments will exit into the interior 30 of the balloon 16. The refrigerant will then undergo a phase change from liquid to gas, simultaneously expanding the balloon and rapidly drawing energy from the surrounding esophageal tissue and causing the tissue to be cooled. The gas may then exhaust though shaft 18 and exit out of the manifold 26 though a port 27. In some other embodiments, the refrigerant supply may require external heating to maintain the desired delivery pressure. The balloon 16 will then expand until contact with the tissue of the esophagus 36 has been made.

The placement of the balloon 16 at the target site and expansion of the balloon is preferably monitored by conventional techniques, such as direct endoscopic visualization. Other endoscopic spectroscopy techniques such as Fluorescence, Raman, or Light Scattering may be useful for identification of atypical esophageal tissue. In order to lower the risk of injury to the esophagus the balloon pressure should be minimized and may be less than 10-psig. Balloon pressure is primarily dependent on the refrigerant flow rate and can be controlled by manipulating the sizes of shaft 18 and/or lumen 20. Pressure can also be controlled though a back-pressure regulator 29, shown in dashed lines in FIG. 1, attached to port 27. Cooling of the esophagus, in particular the atypical esophageal tissue, is typically achieved by evaporation of liquid refrigerant in the balloon 16 which will draw heat away from the esophageal tissue at the target site. In order to ablate or otherwise alter the atypical tissue, it is desirable to cool this tissue until it has frozen. Typically, intracellular ice formation is required for substantial necrosis of the atypical tissue. The target temperature to achieve sufficient intracellular ice formation in the atypical esophageal tissue may be between −25 and −100 C. As undesirable side effects of the cryoablation treatment such as esophageal perforation or stricture may occur if necrosis occurs deeper than the mucosa, the depth of ablation may be controlled by regulating the time that the cooling is applied to the esophagus. Based on typical mucosal thickness of 0.5-2 mm, the required time for ablation may be less than 60 seconds.

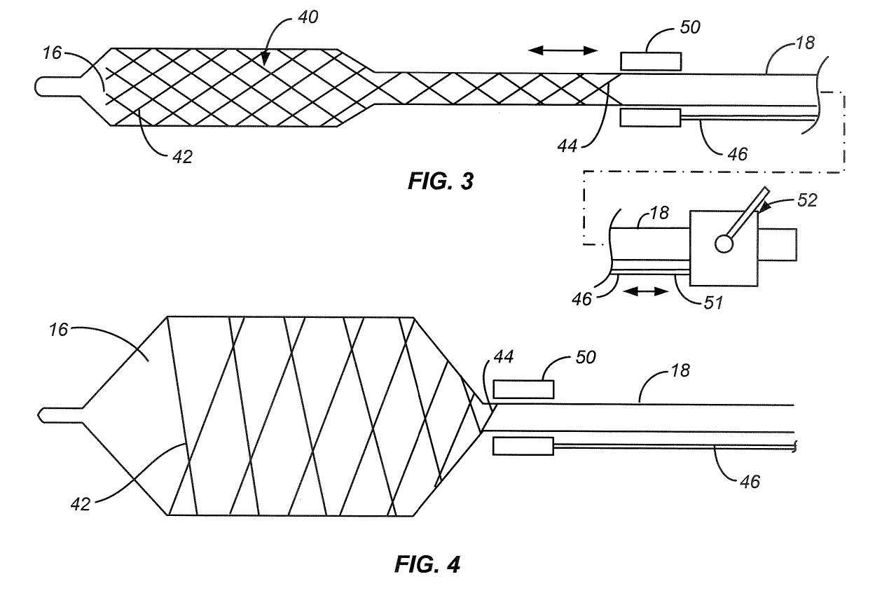

It may be desirable to limit the expansion of the balloon so as to prevent damage to the esophagus. In such cases, a braided structure, see FIGS. 3 and 4, may be used over the outside of the balloon 16. A distal portion 42 of the braided structure may be fixed to the distal end of the catheter assembly 12, that is to the shaft 18 and/or to the balloon 16, and a proximal portion 44 of the braided structure 40, lying proximal to the balloon 16, may be secured to an expansion control rod 46 through a sleeve 50. The proximal end 51 of the control rod 46 may be secured to and may be movable axially with a control element 52, which may be lockable or securable, so that the expansion control rod 46 may move parallel to the placement catheter 18. During use the balloon 16 will be initially inflated to make contact with the esophageal wall 48. This will cause the braided structure 40 to foreshorten which will pull the expansion control rod 46 towards the balloon 16. Once the balloon 16 has reached a desired diameter, the expansion control rod 46 may be secured relative the placement catheter using control element 52; this will prevent additional radial expansion of the braided structure 40 and, in turn, prevent additional dilation of the balloon 16. The use of a radial expansion-limiting tubular braid decreases the compliance of balloon 16. Other techniques for limiting the radial expansion of the balloon 16, such as a coil wrapped around the balloon that when unwound will allow for a progressively larger expansion, may also be used.

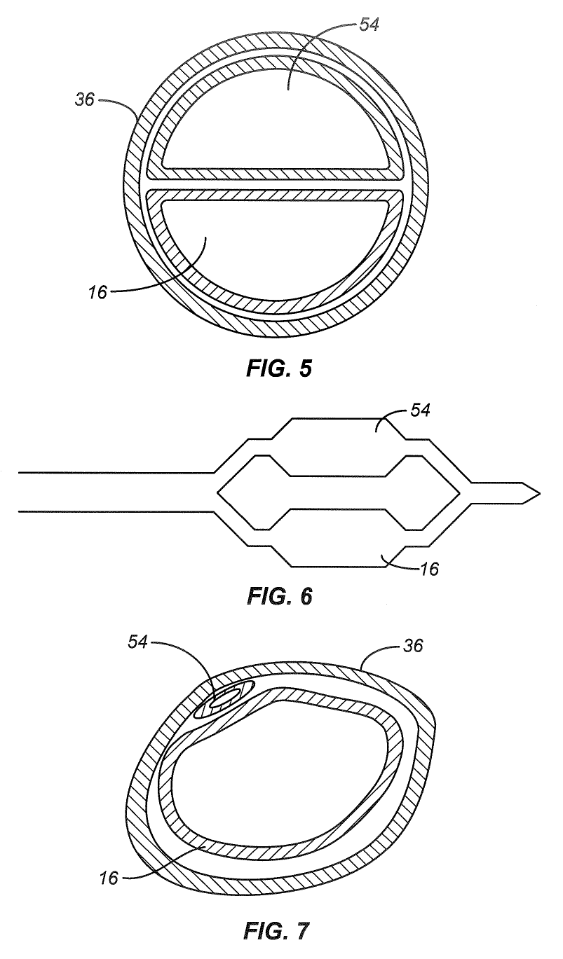

One desirable feature of a balloon-based esophageal tissue treatment system is that the full circumference of the esophagus 36 may be treated simultaneously. However, it may be possible that the desired tissue treatment site extends around only part of the circumference of the esophagus. In this case, it may be desirable to protect portions of the esophagus from the cryoablation. One embodiment of the invention for doing so is illustrated in FIGS. 5-7. In this embodiment, a second balloon 54 may be located adjacent to a first, refrigerant-inflatable balloon 16, balloon 16 acting as a cryoballoon 16. The second balloon 54 may act as an insulating balloon 54. The second, insulating balloon 54 may be constructed of either a compliant material, such as polyurethane or silicone, or non-compliant material, such as PET or nylon, and would typically be manually inflated with an insulating fluid such as air. As shown in broken lines in FIG. 1, a non-refrigerant compressed fluid supply 56, similar to refrigerant supply 14, can be used. Supply 56 may include a nonrefrigerant source 58, such as a compressed air cylinder, and a flow control device 60. Supply 56 may be coupled to second balloon 54 in the same manner as refrigerant supply 14 is coupled to balloon 16. Leaving the second, insulating balloon 54 uninflated may permit the refrigerant-inflatable balloon to expand over substantially the entire circumference. See FIG. 7. Also, a refrigerant may be used in both balloons to provide full circumferential coverage. During operation, the insulating balloon 54 could be inflated in such a way to protect from, for example, 0 to 75% of the circumference of the esophagus. Additional flexibility or control, or both, may be achieved using more than two balloons with the individual balloons being inflatable with the refrigerant and/or with a non-refrigerant. Instead of using separate balloons, a single balloon may include two or more inflatable regions for selective inflation using a refrigerant or a nonrefrigerant. In addition, balloons may be provided or segmented to permit control of cooling of the tissue in a longitudinal or axial direction.

The above descriptions may have used terms such as above, below, top, bottom, over, under, et cetera. These terms are used to aid understanding of the invention are not used in a limiting sense.

While the present invention is disclosed by reference to the preferred embodiments and examples detailed above, it is to be understood that these examples are intended in an illustrative rather than in a limiting sense. It is contemplated that modifications and combinations will occur to those skilled in the art, which modifications and combinations will be within the spirit of the invention and the scope of the following claims.

Any and all patents, patent applications and printed publications referred to above are incorporated by reference.

What is claimed is:

1. A medical device for treating esophageal tissue comprising:
a catheter comprising a distal portion and a lumen;
a balloon mounted to the distal portion to create a catheter assembly;
the balloon having an interior coupled to the lumen, the balloon being placeable within the esophagus of the patient; and
a refrigerant deliverable through the lumen and into the interior of the balloon so to place the balloon into an expanded, cooled state so that the balloon can press against and cool esophageal tissue.

2. The medical device according to claim 1 wherein the balloon is an elastomeric balloon.

3. The medical device according to claim 1 further comprising means for limiting radial expansion of the balloon.

4. The medical device according to claim 1 further comprising:
an expansion-limiting tubular braid surrounding the balloon and having a first end fixed to the catheter assembly and a second end movable along the placement catheter; and
means for fixing the second end at a chosen position relative to the placement catheter so to limit expansion of the tubular braid thereby preventing overexpansion of the balloon.

5. The medical device according to claim 1 wherein the catheter comprises first and second lumens and the balloon interior comprises first and second regions fluidly coupled to the first and second lumens to permit the second region to be free of a refrigerant.

6. The medical device according to claim 5 wherein the balloon comprises first and second balloons defining said first and second regions.

7. The medical device according to claim 5 wherein refrigerant is deliverable into the first region through the first lumen, and further comprising a non-refrigerant deliverable through the second lumen into the second region.

8. The medical device according to claim 1 wherein:
the catheter comprises first and second lumens;
the balloon interior comprises first and second regions fluidly coupled to the first and second lumens; and
a refrigerant deliverable to the first region and a non-refrigerant deliverable to the second region.

9. The medical device according to claim 8 wherein the balloon comprises first and second balloons defining said first and second regions.

10. A method for treating esophageal tissue comprising:
selecting a catheter assembly comprising:
a catheter comprising a distal portion and a lumen; and
a balloon mounted to the distal portion, the balloon having an interior coupled to the lumen;
placing the balloon within the esophagus of a patient;
positioning the balloon at a target site having esophageal target tissue;
delivering a refrigerant through the lumen and into the interior of the balloon thereby:
expanding the balloon to a chosen size; and
cooling the balloon so that the balloon presses against and cools the esophageal target tissue thereby treating the esophageal target tissue.

11. The method according to claim 10 wherein the selecting step is carried out with an elastomeric balloon.

12. The method according to claim 10 further comprising limiting expansion of the balloon.

13. The method according to claim 12 further comprising monitoring the size of the balloon.

14. The method according to claim 13 wherein the monitoring step comprises remotely monitoring the size of the balloon.

15. The method according to claim 13 wherein the remotely monitoring step comprises remotely visualizing the balloon.

16. The method according to claim 12 wherein the expansion limiting step comprises:
surrounding the balloon with an expansion-limiting tubular braid and having a first end fixed to the catheter assembly and a second end movable along the placement catheter; and
fixing the second end to the catheter assembly at a chosen position thereby:
limiting expansion of the tubular braid; and
preventing overexpansion of the balloon.

17. The method according to claim 10 further comprising cooling only a portion of the esophageal tissue in contact with the expanded balloon.

18. The method according to claim 17 wherein:
the catheter assembly selecting step is carried out with a catheter comprising first and second lumens and the balloon interior comprising first and second regions fluidly coupled to the first and second lumens; and
the refrigerant delivery step comprises delivering refrigerant to the first region and a delivering a non-refrigerant to the second region.

19. The method according to claim 18 wherein the catheter assembly selecting step is carried out with the balloon comprising first and second balloons defining said first and second regions.

20. The method according to claim 18 further comprising controlling the expansion of the first and second regions.

21. The method according to claim 20 wherein the expansion controlling step comprises controlling the delivery of at least one of the refrigerant and the non-refrigerant to the first and second regions.
